(19) United States
(12) Patent Application Publication
(10) Pub. No.: US 2002/0151837 A1
(43) Pub. Date: Oct. 17, 2002

(54) Surgical irrigation apparatus and methods for use

(75) Inventor: John I. Shipp, Tullahoma, TN (US)

Correspondence Address: MARTIN & FERRARO, 14500 AVION PARKWAY, SUITE 300, CHANTILLY, VA, 201511101

(73) Assignee: Surgicon Inc.

(21) Appl. No.: 10/123,691

(22) Filed: Apr. 15, 2002

Related U.S. Application Data
(60) Provisional application No. 60/284,361, filed on Apr. 16, 2001.

(51) Int. Cl. A61M 1/00

(52) U.S. Cl. 604/35

(57) ABSTRACT

A surgical irrigation apparatus includes a disposable assembly and a reusable assembly. The disposable assembly includes an irrigation fluid pump and a hand piece having a supply passage for delivery of irrigation fluid to the surgical site. The reusable assembly includes a motor, a power source, and a pressure detector. The pressure detector assists in changing the power being supplied to the motor. The power is transferred from the motor to the pump with a magnetic coupler or other suitable device. The reusable assembly may alternatively include a sound receiver for picking up voice commands or other sounds to change the power being supplied to the motor. The reusable assembly may also alternatively include a controller that is operatively connected to a switch on the hand piece to permit a user to manually change the power being supplied to the motor. Related methods for surgical irrigation are also described.

### RELATED APPLICATIONS

This application claims the benefit of U.S. Provisional Application No. 60/284,361, filed Apr. 16, 2001, incorporated by reference herein.

### BACKGROUND OF THE INVENTION

1. Field of the Invention

The present invention relates generally to the field of surgical suction and irrigation, and more particularly, but not by way of limitation, to an apparatus adapted for use in endoscopic surgery.

2. Description of the Related Art

The use of suction and irrigation devices for both open and endoscopic surgery are well known in the art. U.S. Pat. No. 5,484,402 (hereinafter the '402 patent) describes such a device whereby irrigation fluid is pumped from a source such as a saline bag through a cannula housed in a hand-held probe that is in turn inserted through a trocar for supplying irrigation during laparoscopic surgery. The liquid flow is controlled by a valve in the hand piece which also contains electrical contacts that start the pump simultaneous with the valve opening by completing a circuit connecting a battery pack to the pump motor. Thus, pressurized irrigant is delivered to the surgical field. The switch associated with the irrigation valve is connected by a two conductor electrical cable to the pumping system that contains the battery, motor, and pump housing. Removal of the irrigant, blood, and other such matter is accomplished by opening a second valve in the hand piece thereby connecting a second cannula in the probe to a vacuum source such as a standard hospital vacuum outlet.

The irrigant and all the elements of the system that come into contact with the surgical field must be sterile in order to prevent the spread of infectious diseases. The system described in the '402 patent is a single use device, all the components of which are disposed of after each procedure. This is usually necessary for such devices because of the cost and difficulty encountered in decontamination and re-sterilization. In this respect, hoses and cannula are particularly susceptible to the problems associated with re-sterilization. While the above-described system is useful, it is inadequate because it is too expensive owing to the design details and disposability issues.

U.S. Pat. No. 6,162,194 to Shipp, the disclosure of which is incorporated by reference herein, describes an irrigation/suction system that is inexpensive to manufacture and one that reuses as many of the parts as is practical, combining single use inexpensive parts with multi-use parts that typically do not require decontamination or sterilization prior to the next procedure. The mechanical opening and closing of the irrigation valve is detected by a flow indicator. The flow indicator produces a response that activates or shuts off the pump depending on the valve setting. This allows for pump control without the need for an electrical component in the disposable part of the apparatus.

Often, however, it is desirable to control the flow rate and pressure of the irrigation apparatus. U.S. Pat. No. 6,106,494 (hereinafter the '494 patent) describes a disposable irrigation device that is used predominately in arthroscopy. The apparatus uses a battery operated pump, an inflow tube, an accumulator, a discharge line with a collector container, a static pressure measuring line, and a hand held controller. The controller uses two switches, a printed wiring board, electronic controllers, five pressure transducers, a flow valve, and other complex and expensive devices, all of which are disposed of after a single use. The hand controller has buttons for increasing motor speed. Provisions are made in the hand held controller via an algorithm and appropriate circuitry for automatically adjusting the motor speed to compensate for system changes such as battery voltage variations to keep flow rates and pressures constant. Provisions are also contained in the controller that allow the surgeon to adjust the motor speed up or down.

The device of the '494 patent measures flow rate in the inflow line, flow rate in the discharge line, and the static pressure of the fluid in the body cavity such as a knee joint. The accumulator is used to stop back flow during transit conditions or interruptions in flow, and the control algorithm integrates out certain transits. One purpose of the device is to provide the surgeon with control of the flow rate to the cavity and more importantly the pressure of the irrigant since the pressure is used to expand the joint for certain procedures. The device of the '494 patent is overly complex. Except for transit conditions and leakage of irrigant, the flow in the inflow tube and the discharge tube are virtually identical. Since transits are essentially eliminated in the algorithm and the accumulator, it is unnecessary to measure both flow rates in a well-designed, low-leak system.

What is needed then is an irrigation system that is inexpensive to manufacture and one that reuses as many of the parts as is practical, combining single use inexpensive parts with multi-use parts that, preferably, do not require decontamination or sterilization prior to the next procedure and still allows for complex irrigation tasks such as controlling the flow rate and pressure of the irrigant to a body cavity.

### SUMMARY OF THE INVENTION

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

The present invention in a preferred embodiment is directed to an irrigation system for use in surgery, for example, arthroscopy surgery, such that the principal components, the pump motor and its power source, preferably a battery, and the pressure transducer are reusable and do not require sterilization. Additionally, the present invention allows for complex control of pressure and flow rate from a simple, disposable hand piece. The apparatus of the present invention thus minimizes the disposable components for economic and ecological advantages while maintaining the simplicity of a battery-operated device.

In accordance with the purposes of the present invention, as embodied and broadly described herein, an irrigation system of this invention is provided for use in surgery. The system includes a pump housing having a pump therein for pumping irrigation fluid to a patient, and a passage connected to the pump housing for conducting the irrigation fluid from the pump. The system also includes a hand piece connected to the passage. The hand piece has a mechanism for initiating a flow of the irrigation fluid from the pump to the hand piece. A motor housing having a variable speed motor therein operates the pump. The motor housing is operatively connected to the pump housing. A power supply energizes the motor, and a controller operatively connected to the motor and the power supply regulates power to the motor. The system also includes a pressure transducer housed within at least one of the pump housing and the motor housing. The pressure transducer is operatively connected to the controller for providing pressure data for use in regulating the power supplied to the motor.

In accordance with the purposes of a further embodiment of the present invention, as embodied and broadly described herein, a method of this invention is provided for irrigating a surgical site. The method includes the step of providing an irrigation device having a pump housing, a motor housing, and a pressure transducer housed within at least one of the pump housing and the motor housing. The pump housing has a pump. The motor housing has a motor with a controller associated therewith. The controller is operable to change an amount of power supplied to the motor by a power supply for energizing the motor. The method also includes the steps of pumping irrigation fluid to a patient with the pump; receiving pressure data through the pressure transducer; and changing the amount of the power being supplied to the motor based on the pressure data received.

In accordance with the purposes of another embodiment of the present invention, as embodied and broadly described herein, an irrigation system of this invention is provided for use in surgery. The system includes a pump for pumping irrigation fluid to a patient, and a passage connected to the pump for conducting the irrigation fluid from the pump. A hand piece is connected to the passage and has a mechanism for initiating a flow of the irrigation fluid from the pump to the hand piece. A motor operates the pump. A power supply energizes the motor. A controller operatively connected to the motor and the power supply regulates the power supplied to the motor. A sound receiver is operatively connected to the controller for providing sound data for use in regulating the power supplied to the motor.

In accordance with the purposes of a further embodiment of the present invention, as embodied and broadly described herein, a method of this invention is provided for irrigating a surgical site. The method includes the step of pumping irrigation fluid to a patient with a pump having a motor with a controller associated therewith. The controller is operable to change an amount of power supplied to the motor by a power supply. The method also includes the steps of receiving sound data through a sound receiver operatively connected to the controller; and changing the amount of the power being supplied to the motor based on the sound data received.

In accordance with the purposes of another embodiment of the present invention, as embodied and broadly described herein, an irrigation system of this invention is provided for use in surgery. The system includes a pump housing having a pump therein for pumping irrigation fluid to a patient. A passage connects to the pump housing for conducting the irrigation fluid from the pump. A motor housing having a variable speed motor therein operates the pump. The motor housing operatively connects to the pump housing. A power supply energizes the motor. A controller is housed within the motor housing. The controller is operatively connected to the motor and the power supply for regulating power to the motor.

A hand piece connected to the passage has a mechanism for initiating a flow of the irrigation fluid from the pump to the hand piece. The hand piece has a switch adapted to electronically signal the controller to change the amount of power being supplied to the motor in response to a movement of the switch.

In accordance with the purposes of a further embodiment of the present invention, as embodied and broadly described herein, a method of this invention is provided for irrigating a surgical site. The method includes the step of providing an irrigation device having a hand piece with a switch, a pump housing, and a motor housing. The pump housing has a pump. The motor housing has a motor and a controller. The controller is operable to change an amount of power supplied to the motor by a power supply for energizing the motor. The method also includes the steps of pumping irrigation fluid to a patient with the pump; actuating the switch; and changing the amount of the power being supplied to the motor in response to the actuation of the switch.

It is an object of at least one of the embodiments of the present invention to provide a surgical irrigation apparatus having a disposable assembly and a reusable assembly so that a substantial portion of the apparatus can be reused, thus making the system more economical than previous systems.

Numerous other objects, features and advantages of the present invention will be readily apparent to those skilled in the art upon the reading of the following disclosure when taken in conjunction with the accompanying drawings.

It is to be understood that both the foregoing general description and the following detailed description are exemplary and explanatory only and are not restrictive of the invention, as claimed.

The accompanying drawings, which are incorporated in and constitute a part of this specification, illustrate the embodiments of the invention and together with the description, serve to explain the principles of the invention.

### BRIEF DESCRIPTION OF THE DRAWINGS

FIG. 1 is a side elevational view of a preferred embodiment of the system of the present invention in partial cross section.

FIG. 2 is a block diagram of a pump control circuit of the system of FIG. 1.

FIG. 3 is a side elevational view of another preferred embodiment of the system of the present invention in partial cross section.

FIG. 4 is a block diagram of a pump control circuit of the system of FIG. 3.

FIG. 5 is a side elevational view of yet another preferred embodiment of the system of the present invention in partial cross section.

FIG. 6 is a block diagram of a pump control circuit of the system of FIG. 5.

### DETAILED DESCRIPTION OF THE DRAWINGS

Reference will now be made in detail to the present preferred embodiments of the invention, examples of which are illustrated in the accompanying drawings. Wherever possible, the same reference numbers will be used throughout the drawings to refer to the same or like parts.

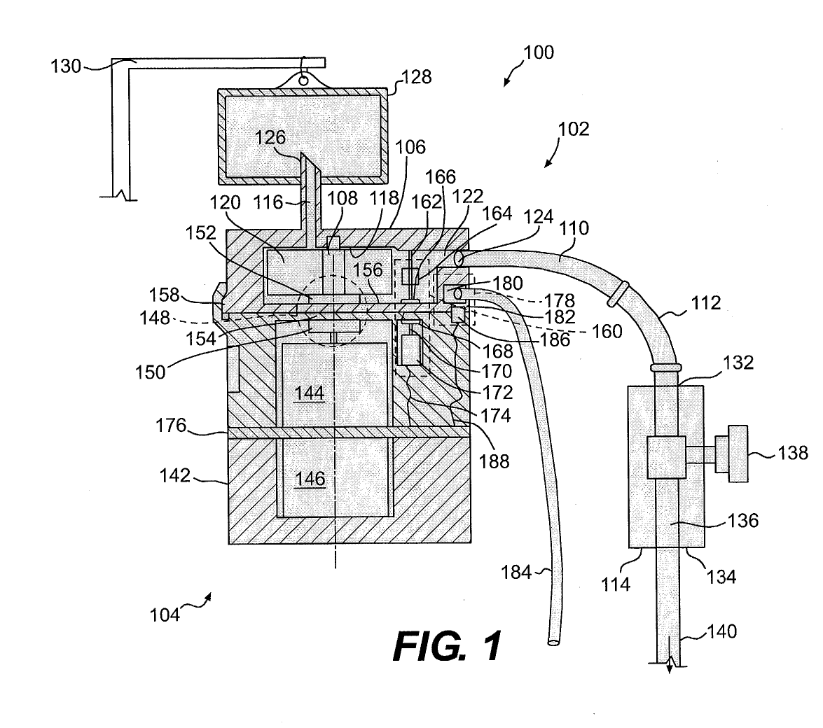

As shown in the drawings and particularly to FIG. 1, a preferred embodiment of a surgical irrigation apparatus of the present invention is generally referred to by the numeral 100. Apparatus 100 includes a disposable assembly 102 and a reusable assembly 104.

Disposable assembly 102 preferably includes a pump housing 106 having an irrigation fluid pump 108 therein, a hose 110, an accumulator 112, and a hand piece 114. Pump 108 has a suction inlet 116 leading to a pump chamber 118 having a centrifugal pump impeller 120 rotatably disposed therein. Chamber 118 in turn leads to an outlet passage 122, and thereafter to a discharge outlet 124. Suction inlet 116 of pump 108 preferably includes a bayonet-type connector 126 that is adapted for insertion into an irrigation fluid supply bag 128 in a conventional manner. Irrigation fluid supply bag 128 is a conventional irrigation fluid bag that is suspended from a support pole 130 as typically used in an operating room.

Discharge outlet 124 of pump 108 is connected to flexible discharge hose 110. Hose 110 connects accumulator 112 to discharge outlet 124 and hand piece 114. Accumulator 112 is preferably designed to integrate out unwanted flow transits by providing a flexible plenum chamber to discourage back flow of irrigant to pump 108 by providing a check valve.

Hand piece 114 has a fluid inlet 132, a fluid outlet 134, and a passage 136 therebetween. Hand piece 114 also has a valve 138 that is preferably manually adjustable and actuatable and controls the flow of irrigation fluid through passage 136 and into a patient inflow tube 140. Valve 138 is preferably a combination continuously variable flow valve and trumpet (momentary) valve. Valve 138 may be set for a particular flow that can be momentarily over-ridden by the momentary action of a standard trumpet valve arrangement. Valve 138 may also be designed to increase or decrease flow in response to the momentary action.

Inflow tube 140 extends from fluid outlet 134 of hand piece 114 to a surgical site accessed by an entry lumen in a trocar inserted into a body cavity such as for example a knee joint. Irrigant enters the body cavity and flows through an exit lumen in the trocar to a discharge collection chamber.

Reusable assembly 104 includes a motor housing 142 preferably having a motor 144 and a power source 146 therein. Motor 144 is preferably a variable speed electric motor. Power source 146 is preferably a battery, which may be a re-chargeable or primary battery. It will be appreciated that power source 146 may be external to motor housing 142. For example, power source 146 may be a standard AC obtained from a conventional wall outlet accessible through a plug-in electric cord.

Apparatus 100 preferably includes a device 148 for transferring power between reusable assembly 104 and disposable assembly 102. Device 148 transfers mechanical power from motor 144 to impeller 120 of pump 108 while isolating irrigation fluid flowing through pump 108 from contact with reusable assembly 104. Device 148 preferably includes a magnetic coupling having a first magnet 150 and a second magnet 152. First magnet 150 is connected to motor 144 and is driven thereby. Second magnet 152 is connected to impeller 120 of pump 108. The magnetic field from first magnet 150 extends through top wall 154 of motor housing 142 and interacts with the magnetic field of second magnet 152 extending through bottom wall 156 of pump housing 106. Walls 154, 156 space apart first and second magnets 150,152, respectively, and also separate reusable assembly 104 from disposable assembly 102 so that sterile irrigation fluid flowing through pump 108 is not contaminated by any part of reusable assembly 104.

A quick connect 158 is preferably provided for connecting reusable assembly 104 to disposable assembly 102 in an efficient, reliable and easy manner in the operating room. Quick connect 158 can take a variety of different forms such as disclosed in U.S. Pat. No. 6,162,194. In use, for example, a nurse handling sterilized components may hold disposable assembly 102, while reusable assembly 104 is held by a nurse not handling sterilized components. Both nurses acting together may quickly connect the components. One of the advantages of the present invention is that the reusable components do not have to be sterilized between usages because they do not come in contact with the irrigation fluid that is being provided to the surgical field.

As shown in FIG. 1, apparatus 100 preferably includes a fluid flow detector 160 for detecting and measuring the flow rate of the irrigation fluid being pumped through pump 108. Fluid flow detector 160 preferably includes a magnetic coupling having a first magnet 162 and a second magnet 164. First magnet 162 is connected to a shaft 166 having an impeller 168 which is disposed in discharge passage 122 of pump 108. Impeller 168 is preferably arranged and constructed so that the flow of irrigation fluid through pump 108 turns impeller 168. Second magnet 164 is connected to a shaft 170 of an electromechanical sensor 172. The magnetic field from first magnet 162 extends through bottom wall 156 of pump housing 106 to interact with the magnetic field of second magnet 164 extending through top wall 154 of motor housing 142. Thus, as impeller 168 rotates, the magnetic coupling between first and second magnets 162, 164, respectively, act to rotate shaft 170 to cause sensor 172 to produce a signal. The signal from sensor 172 is carried through a line 174 to a microprocessor controller 176, described in more detail below.

It will be appreciated that other fluid flow detectors may be used. For example, U.S. Pat. No. 6,162,194 to Shipp describes a flow detector constructed to use a light source. The sensor senses a light signal that passes through a transparent window and is correlated to the flow of fluid through the discharge passage. Another exemplary flow detector may be adapted to sense a pressure change within the pump.

Apparatus 100 also preferably includes a static pressure detector 178 that is at least in part reusable. In a preferred embodiment, pressure detector 178 includes a static pressure chamber 180 preferably housed within pump housing 106. Static pressure chamber 180 is preferably a water-tight compartment having a resilient membrane 182 proximate bottom wall 156 of pump housing 106. A static pressure line 184, preferably made of a surgical grade tubing, provides a connection between static pressure chamber 180 and a location where it is desired to measure the pressure. For example, pressure line 184 may be connected to a trocar for measuring the pressure within a patient cavity.

Pressure detector 178 also preferably includes a pressure transducer 186 at least in part within motor housing 142. Pressure transducer 186 is preferably located proximate top wall 154 of motor housing 142 and is adapted to interact with membrane 182 to measure the pressure within pressure chamber 180. Signals from pressure transducer 186 are carried via line 188 to microprocessor 176. By including at least a portion of pressure transducer 186 within motor housing 142 and making it reusable, numerous advantages are achieved, one of the most important being a reduction in total manufacturing costs.

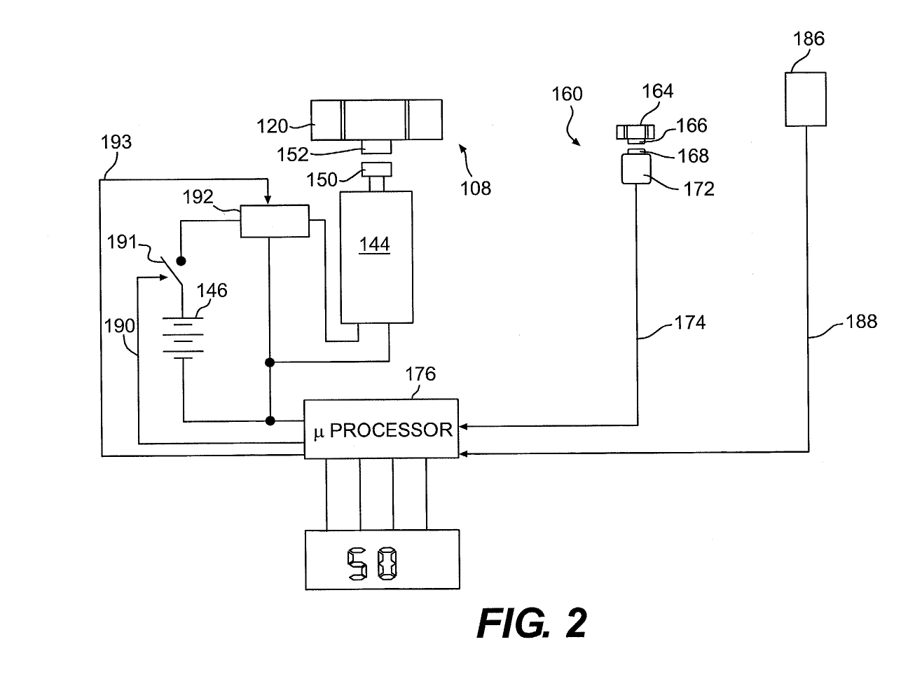

As shown in FIG. 2, microprocessor 176 preferably contains software having an algorithm adapted to activate and deactivate motor 144 through a control line 190 connected to a switch 191. Microprocessor 176 is also preferably connected to a motor control circuit 192 via a control line 193. Motor control circuit 192 is adapted to regulate the power supplied to motor 144 at the command of microprocessor 176, which interprets the flow and pressure data from flow and pressure detectors 160,178, respectively. Although illustrated as two components in FIG. 2, it will be appreciated that microprocessor 176 and motor control circuit 192 may exist as a single component.

A timer in microprocessor 176 preferably keeps track of the battery use time and displays, preferably on a liquid crystal display, the number of procedures remaining prior to battery depletion. Preferably the battery delivers a near constant voltage over the discharge cycle, though other batteries can be accommodated with the addition of current and voltage monitoring circuits.

In use, reusable assembly 104 is connected to disposable assembly 102, for example, through use of quick connect 158. When apparatus 100 is not in use, motor 144 is off and valve 138 is preferably in the closed position. To activate motor 144, the surgeon opens valve 138 and the force of gravity acting upon the irrigation fluid in fluid source 128 causes the irrigation fluid to begin flowing downward through pump 108 and through supply hose 110 to hand piece 114. Very rapidly, upon the beginning of the flow, fluid flow detector 160 detects the flow of irrigation fluid through pump 108. When the irrigation fluid first begins to flow through outlet passage 122 of pump 108, it rotates impeller 164 of flow detector 160, which in turn rotates first magnet 166. The rotation of first magnet 166 causes second magnet 168 to rotate, thus rotating an electromechanical portion of sensor 172 and generating an electrical flow indication signal representative of a flowing fluid. The electrical signal is communicated to microprocessor 176 over electrical line 174.

Microprocessor 176 senses the flow-indicating signal from sensor 172 and sends a signal over control line 190 to switch 191 causing switch 191 to close, thus completing a circuit between motor 144 and battery 146 and causing motor 144 to rotate. In a preferred embodiment, apparatus 100 can detect fluid flow and will typically turn on motor 144 within less than 2.0 seconds, and more preferably less than 0.1 seconds of the opening of valve 138.

When motor 144 rotates, the rotational motion is carried though to pump impeller 170 via first and second magnets 150, 152, respectively, to cause pump impeller 120 to turn and begin pumping irrigation fluid under pressure from pump 108 to hand piece 114. The flow rate of the fluid is preferably determined by the opening position of valve 138 and an initial predetermined motor speed.

During the time that the irrigation fluid is flowing through pump 108, the irrigation fluid, preferably sterile, is isolated from reusable assembly 104 by walls 154, 156 which separate magnets 150, 152, respectively, so that the fluid will remain uncontaminated.

While pump 144 is delivering irrigant under pressure at a flow rate set by valve 138, microprocessor 176, using inputs from flow detector 160 and pressure transducer 186, communicates with motor controller circuit 192 via control line 193 to regulate the power to motor 144 and thus maintain the system operating parameters at a desired predetermined level. The algorithm stored in microprocessor 176 allows the surgeon to change the desired power level by signaling microprocessor 176 using a predetermined code. This may be accomplished, for example, by initiating a transit in the flow with short interrupts of the flow using the biased trumpet action of valve 138. For example only, to call for a decrease in power, the surgeon, with a rapid momentary action, slows or stops the flow using the trumpet action of valve 138. The resulting flow and pressure transit is seen as input to microprocessor 176 via lines 174 and 188, respectively. For example, a single transit of a given time span may be interpreted by the algorithm in microprocessor 176 as a call to decrease the power to motor 144. Double transits caused by the surgeon quickly double-stroking the trumpet action of valve 138 may be used as a call to increase the power. The number of transits necessary to increase or decrease the power to motor 144 can be programmed as a matter of preference.

Flow sensor 160 may also be adapted to sense when there is no flow, such as when valve 138 is closed, and thus send a signal to open switch 191 and turn off motor 144. The algorithm stored in microprocessor 176 differentiates between system transits toward low flow and system shut off by using a pre-selected time interval chosen by the surgeon for which no flow occurs.

After the surgical procedure, disposable assembly 102 may be easily detached from reusable assembly 104 and disposed of. Thereafter, reusable assembly 104 may be connected to another disposable assembly 102 for supplying irrigation fluid for another surgical procedure.

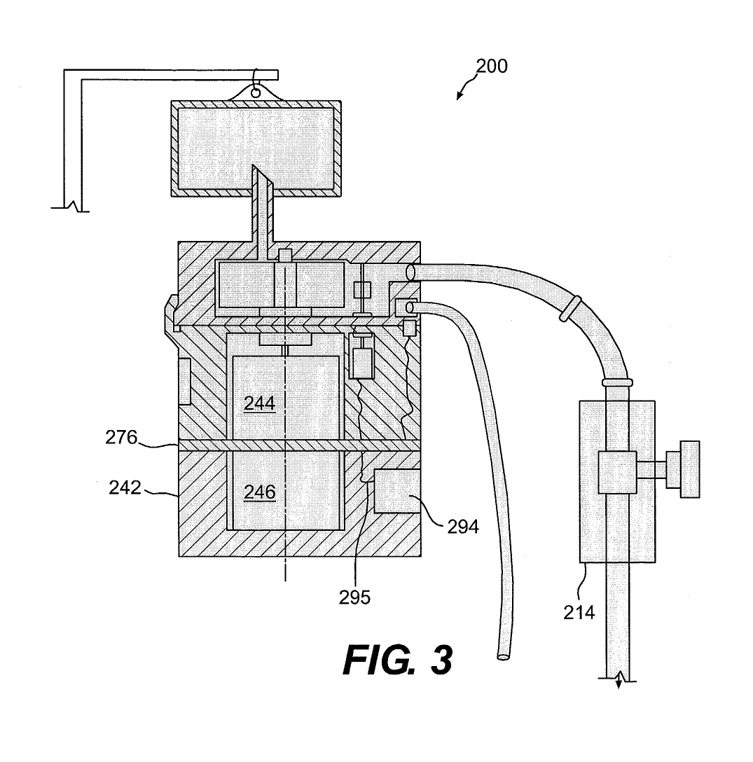

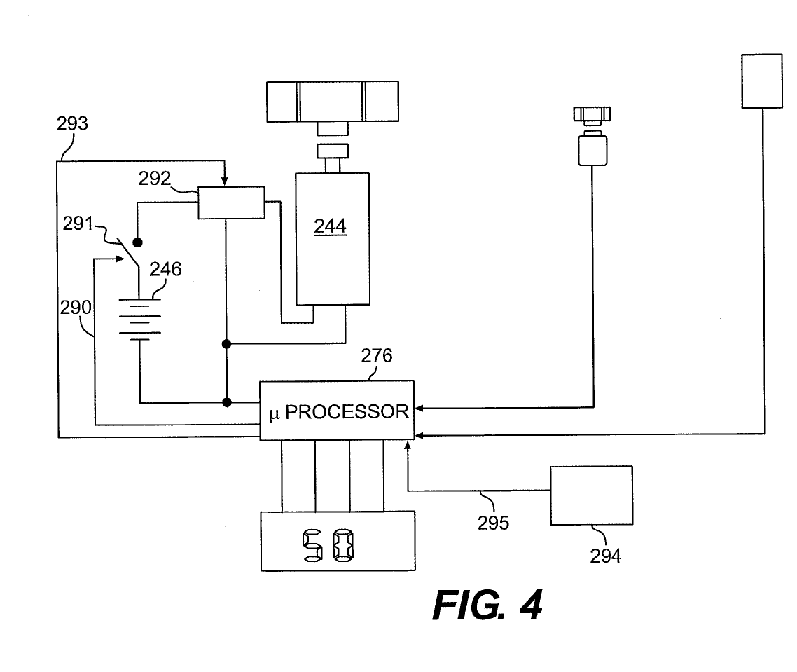

FIGS. 3 and 4 show another preferred embodiment of a surgical irrigation apparatus of the present invention generally referred to by the numeral 200. Apparatus 200 is similar to apparatus 100 except that it preferably includes a sound receiver 294 operatively connected to microprocessor 276. Sound receiver 294 permits microprocessor 276 to receive and process sound data to control the power supplied to motor 244. Sound receiver 294 transforms sounds into an electrical signal communicated to microprocessor 276 via line 295. Microprocessor 276 has a memory preferably containing a conventional voice or sound recognition software that delivers an appropriate output signal via control line 293 to motor controller 292 to decrease, increase, or otherwise regulate the power to motor 244.

In one preferred embodiment, the sound data may include voice commands. To process voice commands, microprocessor 276 may include conventional speech recognition software so that speech from the surgeon is interpreted and coded. An example of speech recognition software is described in U.S. Pat. No. 6,031,526 to Shipp. Thus, upon an appropriate voice command, power to motor 244 may be increased or decreased.

Alternately, the sound data may include one or more mechanical sounds. For example, hand piece 114 may include a characteristic sound-producing device in communication with sound receiver 294 such that when the sound-producing device is activated in predetermined patterns, the pump power may be changed. For example, with a single characteristic click, a signal may be sent to control circuit 292 to increase the power supplied to motor 244. A double click may be used to decrease power supplied to motor 244. Regardless of the type of sound data used, microprocessor 276 may have software adapted to discriminate between pump control sounds and other operating room sounds where so desired.

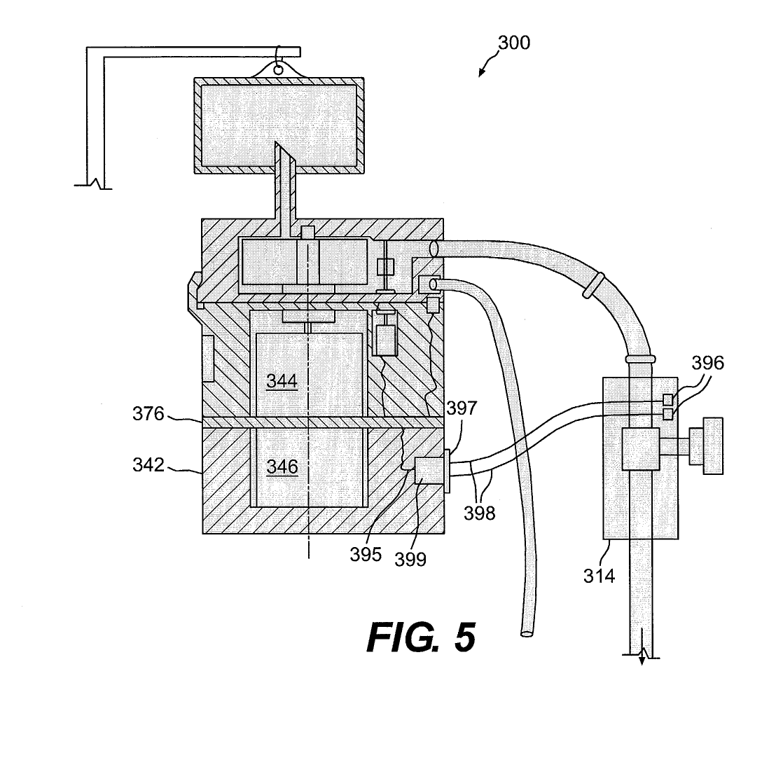

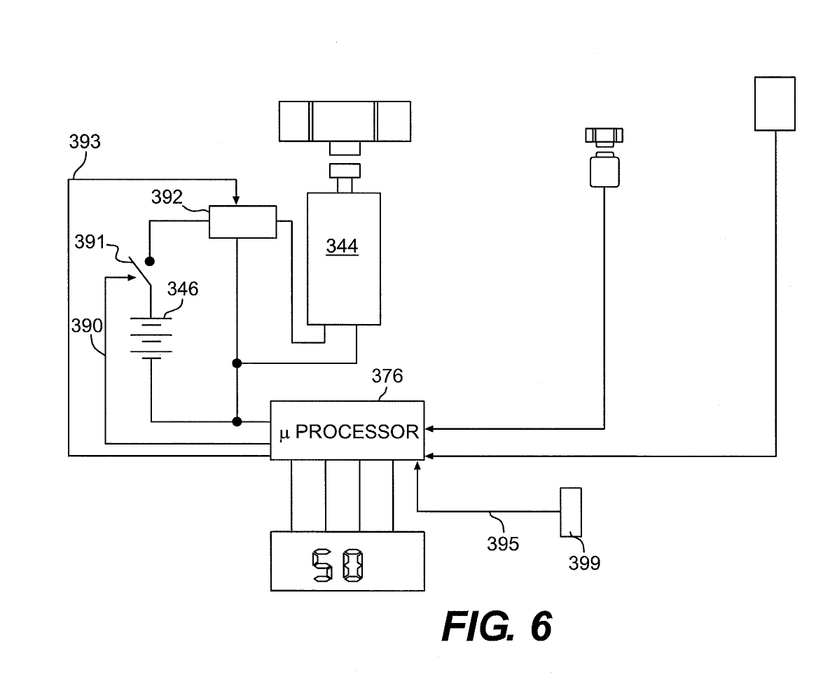

FIGS. 5 and 6 show another preferred embodiment of a surgical irrigation apparatus of the present invention generally referred to by the numeral 300. Apparatus 300 is similar to apparatus 100 except that the amount of power supplied to motor 344 may be changed directly by the surgeon using one or more buttons and/or switches. Switches 396 are preferably positioned on hand piece 314 to allow the surgeon easy control of the power supplied to motor 344. Switches 396 are connected to an electrical cable 397 that terminates at electrical connector 398. Electrical connector 398 is in turn plugged into a mating connector 399 preferably located on motor housing 342. Mating connector 399 is in electrical communication with microprocessor 376. Signals indicating the opening and closuring of switches 396 are delivered to microprocessor 376 via line 395. An algorithm stored in microprocessor 376 decodes the inputs from the switches. In a preferred embodiment, one switch closure may be used to indicate a call for decreased power to motor 344 while the other switch closure may be used to indicate a call for increased power to motor 344. Thereafter, microprocessor 376 instructs motor controller 392 via control line 393 to decrease or increase the power to motor 344 according to the signals received from switches 396.

In use, the distal end of sterile electrical cable 397 is attached to switches 396. The proximal end of cable 397 is attached to electrical connector 398. Upon installation of disposable sterile assembly 302 with reusable assembly 304, electrical cable 397 is extended beyond the sterile zone of the operating table and attached to mating connector 399. This does not compromise the sterile field and allows power to be increased or decreased to motor 344 by the surgeon's actuation of switches 396.

Thus, it is seen that the apparatus and methods of the present invention readily achieve the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention as defined by the appended claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

I claim:

1. An irrigation system for use in surgery, said system comprising:
a pump housing having a pump therein for pumping irrigation fluid to a patient;
a passage connected to said pump housing for conducting the irrigation fluid from said pump;
a hand piece connected to said passage, said hand piece having a mechanism for initiating a flow of the irrigation fluid from said pump to said hand piece;
a motor housing having a variable speed motor therein for operating said pump, said motor housing being operatively connected to said pump housing; a power supply for energizing said motor;
a controller operatively connected to said motor and said power supply for regulating power to said motor; and
a pressure transducer housed within at least one of said pump housing and said motor housing, said pressure transducer being operatively connected to said controller for providing pressure data for use in regulating the power supplied to said motor.

2. The system of claim 1, wherein said pressure transducer is housed within said motor housing.

3. The system of claim 2, wherein said motor housing is detachable from said pump housing.

4. The system of claim 2, wherein said pump housing includes a static pressure chamber in communication with said pressure transducer.

5. The system of claim 4, wherein said pump housing includes a bottom surface having a flexible membrane along at least a portion thereof, said static pressure chamber being located proximate said membrane.

6. The system of claim 4, wherein said system includes a static pressure tube adapted for connection between said static pressure chamber and a trocar.

7. The system of claim 1, wherein said motor housing is reusable.

8. A method of irrigating a surgical site, said method comprising the steps of:
providing an irrigation device having a pump housing, a motor housing, and a pressure transducer housed within at least one of the pump housing and the motor housing, the pump housing having a pump, the motor housing having a motor with a controller associated therewith, the controller being operable to change an amount of power supplied to the motor by a power supply for energizing the motor;
pumping irrigation fluid to a patient with the pump;
receiving pressure data through the pressure transducer; and
changing the amount of the power being supplied to the motor based on the pressure data received.

9. The method of claim 8, wherein the step of providing an irrigation device includes providing the pressure transducer in the motor housing.

10. The method of claim 8, further comprising the step of detaching the motor housing from the pump housing.

11. The method of claim 10, further comprising the step of attaching the motor housing to a different pump housing.

12. The method of claim 8, wherein the pump housing includes a static pressure chamber and the step of receiving pressure data includes measuring the pressure of the static pressure chamber of the pump housing.

13. An irrigation system for use in surgery, said system comprising:
a pump for pumping irrigation fluid to a patient;
a passage connected to said pump for conducting the irrigation fluid from said pump;
a hand piece connected to said passage, said hand piece having a mechanism for initiating a flow of the irrigation fluid from said pump to said hand piece;
a motor for operating said pump;
a power supply for energizing said motor;
a controller operatively connected to said motor and said power supply for regulating the power supplied to said motor; and
a sound receiver operatively connected to said controller for providing sound data for use in regulating the power supplied to said motor.

14. The system of claim 13, wherein said controller includes a memory adapted to store a software program for interpreting the sound data provided by said sound receiver for use in regulating the power supplied to said motor.

15. The system of claim 14, wherein said software program is adapted to interpret a selected voice command and produce a signal based on the selected voice command to regulate the power supplied to said motor.

16. The system of claim 14, wherein said software program is adapted to recognize a selected mechanical sound and produce a signal based on the selected mechanical sound to regulate the power supplied to said motor.

17. The system of claim 16, wherein said software program is adapted to recognize a mechanical sound produced by a mechanism connected to said hand piece.

18. The system of claim 13, wherein said motor is a variable speed motor.

19. A method of irrigating a surgical site, said method comprising the steps of:
pumping irrigation fluid to a patient with a pump having a motor with a controller associated therewith, the controller being operable to change an amount of power supplied to the motor by a power supply;
receiving sound data through a sound receiver operatively connected to the controller; and
changing the amount of the power being supplied to the motor based on the sound data received.

20. The method of claim 19, wherein the sound data received is a selected voice command, further comprising the steps of interpreting the voice command and producing a signal representative of the voice command to change the amount of power being supplied to the motor.

21. The method of claim 19, wherein the sound data received is a selected mechanical sound, further comprising the steps of recognizing the mechanical sound and producing a signal representative of the mechanical sound to change the amount of power being supplied to the motor.

22. The method of claim 21, further comprising the step of producing the selected mechanical sound from a hand piece operatively connected to the pump.

23. The method of claim 19, wherein the step of changing the amount of power includes at least one of activating or deactivating the motor.

24. The method of claim 19, wherein the motor is a variable speed motor and the step of changing the amount of power includes at least one of increasing or decreasing the amount of power being supplied to the variable speed motor.

25. An irrigation system for use in surgery, said system comprising:
a pump housing having a pump therein for pumping irrigation fluid to a patient;
a passage connected to said pump housing for conducting the irrigation fluid from said pump;
a motor housing having a variable speed motor therein for operating said pump, said motor housing being operatively connected to said pump housing; a power supply for energizing said motor;
a controller housed within said motor housing, said controller being operatively connected to said motor and said power supply for regulating power to said motor;
a hand piece connected to said passage, said hand piece having a mechanism for initiating a flow of the irrigation fluid from said pump to said hand piece, said hand piece having a switch adapted to electronically signal said controller to change the amount of power being supplied to said motor in response to an actuation of said switch.

26. The system of claim 25, further comprising a cable connecting said hand piece to said motor housing, said cable being adapted to deliver the signal from said switch to said controller.

27. The system of claim 26, wherein said cable has an end having an electrical connector and said motor housing has a mating connector operatively connected to said controller, said electrical connector being releaseably attachable to said mating connector.

28. The system of claim 25, wherein said motor housing is detachable from said pump housing.

29. The system of claim 25, further comprising a second switch, at least one of said switches being adapted to signal said controller to increase power to said motor, the other of said switches being adapted to signal said controller to decrease power to said motor.

30. A method of irrigating a surgical site, said method comprising the steps of:
providing an irrigation device having a hand piece with a switch, a pump housing, and a motor housing, the pump housing having a pump, the motor housing having a motor and a controller, the controller being operable to change an amount of power supplied to the motor by a power supply for energizing the motor;
pumping irrigation fluid to a patient with the pump; actuating the switch; and
changing the amount of the power being supplied to the motor in response to the actuation of the switch.

31. The method of claim 30, wherein the irrigation device includes a cable connecting the hand piece to the motor housing and the step of actuating the switch includes sending a signal through the cable to the controller in the motor housing.

32. The method of claim 31, wherein the cable is detachable from the motor housing, further comprising the step of detaching the cable from the motor housing.

33. The method of claim 30, wherein the motor housing is detachable from the pump housing, further comprising the step of detaching the motor housing from the pump housing.
